(19) United States
(12) Patent Application Publication
(10) Pub. No.: US 2025/0221614 A1
(43) Pub. Date: Jul. 10, 2025

(54) HYPER-EXCITATION HYPERSPECTRAL IMAGING SYSTEM

(71) Applicant: The George Washington University, Washington, DC (US)

(72) Inventor: Narine Sarvazyan, Lambertville, NJ (US)

(73) Assignee: The George Washington University, Washington, DC (US)

(21) Appl. No.: 19/065,051

(22) Filed: Feb. 27, 2025

Related U.S. Application Data
(63) Continuation-in-part of application No. PCT/US2023/031963, filed on Sep. 5, 2023.
(60) Provisional application No. 63/403,355, filed on Sep. 2, 2022.

(51) Int. Cl. A61B 1/06; A61B 1/04

(52) U.S. Cl. CPC A61B 1/0638; A61B 1/043; A61B 1/046; A61B 1/0646; A61B 1/0661

(57) ABSTRACT

A hyper-excitation hyperspectral imaging (HE-HSI) system. The system represents a technology-empowered expansion of spectroscopy into the imaging domain. The system includes optical components such as tunable filters, light sources, hyperspectral cameras, and advanced data processing algorithms. It addresses one of the major challenges of the traditional hyperspectral imaging approach such as the need to identify specific excitation and/or emission wavelengths where spectral differences are the most pronounced and consistent. The latter is particularly challenging for spectrally complex surfaces of human organs which can be quite variable between individuals. The HE-HSI system collects information across the entire UV-VIS-IR range by collecting both excitation and emission data from each pixel of an image. HE-HSI yields a computationally rich 4D data array that includes both fluorescence and reflectance profiles. The new modality identifies additional targets previously indistinguishable by traditional hyperspectral imaging while reducing the amount of effort needed to find the optimal illumination and acquisition wavelengths. The system further includes portable HE-HSI devices for dermatological applications and HE-HSI-based endoscopic and percutaneous imaging devices as novel surgical tools.

### CROSS-REFERENCE TO RELATED APPLICATIONS

This application is a continuation-in-part of PCT/US2023/031963, filed Sep. 5, 2023, which claims the benefit of priority of U.S. Application Ser. No. 63/403,355 filed on Sep. 2, 2022, the contents of which are relied upon and incorporated herein by reference in its entirety.

### BACKGROUND

Hyperspectral imaging (HSI) is a technology that captures spectral information across multiple wavelengths from each pixel of an image. That information facilitates the identification and classification of objects, materials, or areas of an image based on their spectral properties. Contrary to conventional color imaging systems that record intensity from red, green, and blue bands, HSI creates an extensive array of nearly contiguous spectrum, thereby enabling the detection and categorization of small differences in spectral properties of the target, including object's diffuse reflection, absorption or autofluorescence. One limitation of HSI is that in its current form it collects only a small subset of useful spectral information.

Excitation-emission matrix (EEM) spectroscopy is a fluorescence technique that measures the intensities of emission spectra at different excitation wavelengths. It generates a three-dimensional data array with the x-axis representing excitation wavelengths, the y-axis emission wavelengths, and the z-axis the fluorescence intensity. The locations of emission peaks on the EEM landscape provide identification of fluorophores while the peak's intensities give quantitative information. EEM provides rapid fingerprinting of complex fluorescent samples for various analytical applications. One limitation of traditional EEM is that it only collects data from a single point and not an image.

The sensitivity of sensors within snapshot cameras has improved dramatically with the expansion of applications such as drone-based imaging and surveying, which demand integration times suitable for video framerates. Similar developments are occurring for data processing algorithms and optical tuning of filters and light sources. Therefore, the duration of processes that took minutes can be now shortened to a fraction of a second.

### SUMMARY

One embodiment of the current disclosure is a near-real time Hyper-Excitation Hyperspectral (HE-HSI) imaging system and method. The imaging system merges HSI and EEM-based approaches into a new modality referred here as Hyper-Excitation Hyperspectral Imaging or HE-HSI. HE-HSI resolves limitations of both approaches, i.e., traditional HSI and EEMs, since it collects a complete set of spectral information including fluorescence, from each pixel of an image. The use of HE-HSI resolves one of the major challenges of traditional HSI, which is the need to identify specific excitation or emission wavelengths where spectral differences are consistent and/or pronounced. Once such areas within excitation-emission space are identified, the amount of required spectral information to be acquired can dramatically decrease. Moreover, target-specific mathematical equations can then be developed for discrimination of surgical targets, improving diagnostic output, or designing affordable LED driven surgical devices.

This summary is not intended to identify all essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIGS. 1(a), (b) show two basic hardware configurations to acquire HE-HSI datasets.

FIG. 1(c) illustrates the information contained within a HE-HSI dataset, with each pixel holding a full set of EEM values.

FIGS. 2(a), (b), (c) show diagrams of HE-HSI systems for fast acquisition of reflectance and fluorescence spectra using snapshot hyperspectral cameras which acquire multiple spectral planes simultaneously. The configuration shown in FIG. 2(a) can be designed to have insertable components or to switch between the two configurations shown in FIG. 2(b) and FIG. 2(c).

FIGS. 3(a), (b) show diagrams of HE-HSI systems that use hyperspectral cameras that acquire multiple spectral planes sequentially. The configuration shown in FIG. 3(a) can be designed to have insertable components or to switch between the two paths shown. FIG. 3(b) shows an alternative configuration that employs a single excitation tunable light source with wavelength-dependent gain modulation. The latter enables to reduce camera gain when excitation wavelength is close to the emission wavelength.

FIG. 4 shows a general concept of HE-HSI data processing.

FIG. 5 shows the key principle behind the increased sensitivity of HE-HSI approach.

FIGS. 6(a), 6(b) illustrate the increased sensitivity of HE-HSI system compared to traditional HSI based on single wavelength excitation.

FIGS. 7(a), (b), (c) show EEMs from native (FIG. 7(a)), ablated (FIG. 7(b)), and scarred (FIG. 7(c)) rat heart tissue, where the arrows point for NADH signal (or absence thereof) and collagen accumulation.

FIGS. 7(d), 7(e) shows spectral profiles derived from the EEMs above using 50 nm (FIG. 7(d)) or 100 nm (FIG. 7(e)) offsets between the excitation and emission wavelengths.

FIGS. 8(a)-8(d) show unmixing results and analysis of 3D vs. 4D HSI configurations.

The figures show illustrative embodiment(s) of the present disclosure. Other embodiments can have components of different scale. Like numbers used in the figures may be used to refer to like components. However, the use of a number to refer to a component or step in a given figure has a same structure or function when used in another figure labeled with the same number, except as otherwise noted.

### DETAILED DESCRIPTION

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure.

### Data Acquisition

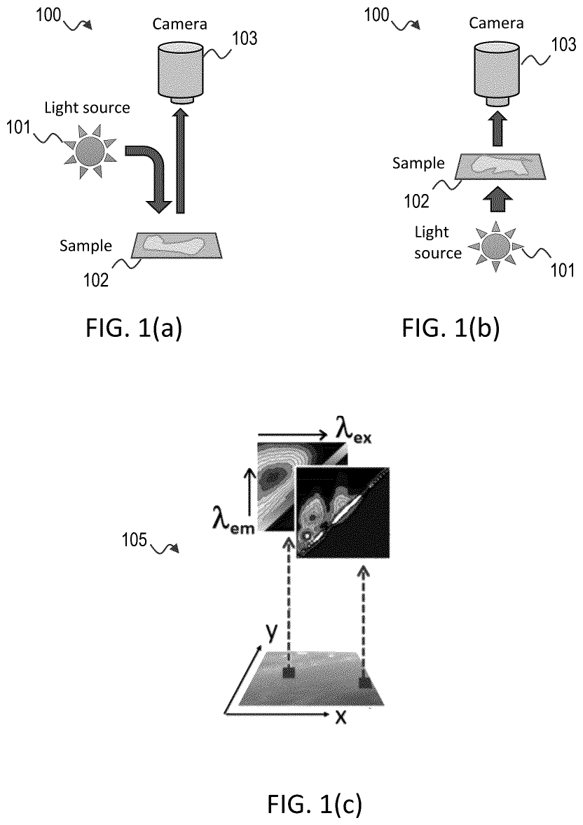

Turning to the drawings, FIGS. 1-3 show illustrative, non-limiting example embodiments of the disclosure depicting different configurations of a hyperexcitation hyperspectral imaging (HE-HSI) system that collects both reflectance and fluorescence information. It is noted that multi-excitation hyperspectral imaging (ME-HSI) is a subset of HE-HSI. The two possible hardware configurations of the HE-HSI imaging system 100 are shown in FIG. 1(a) and FIG. 1(b), which include a variable light source 101, a sample 102 and an imaging device such as for example a camera 103. The variable light source can produce different excitation wavelengths and can be, for example, (1) a broadband light source followed by tunable filter or a filter wheel, (2) a collection of switchable/rotatable light sources, (3) a tunable light source. The camera 103 can be, for example, (1) a snapshot hyperspectral camera that acquires multiple spectral planes at once, (2) a hyperspectral camera with imbedded tunable filter, or (3) an assembly of black/white cameras with a filter unit in front of it.

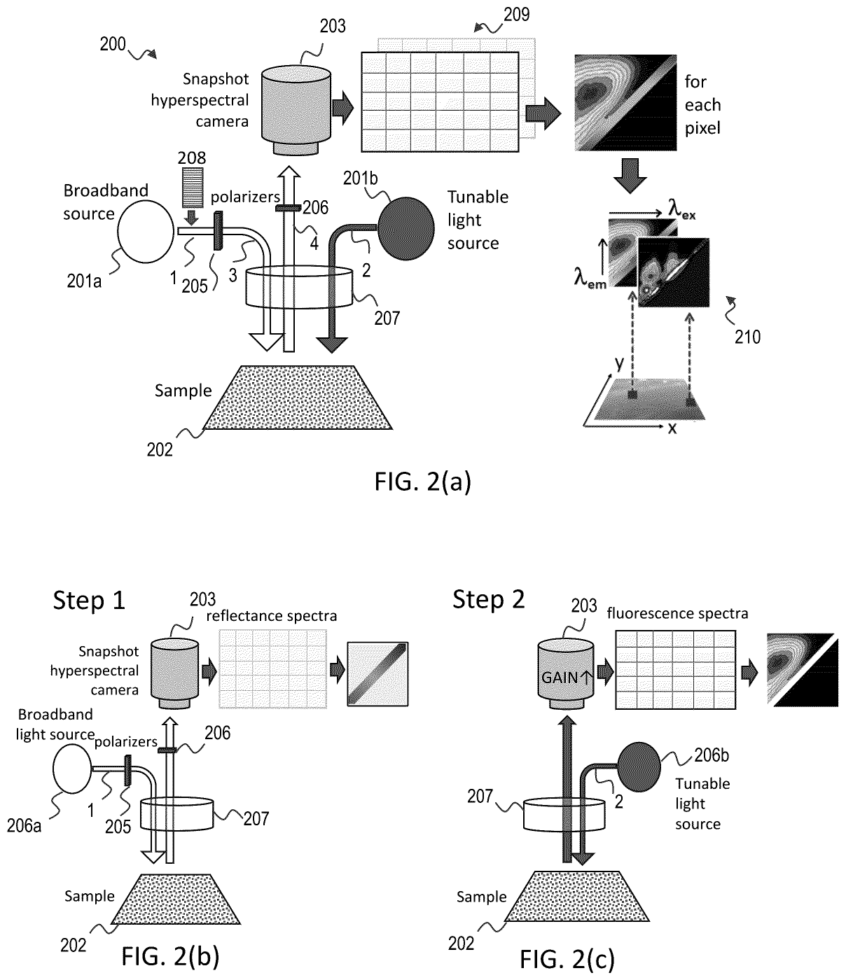

FIG. 1(a) shows a configuration to collect HE-HSI data from a surface of a non-transparent object. Light from source 101 is directed to the top surface of the sample 102, while light coming from the sample 102 is captured by the camera 103. FIGS. 2-7(b) are in accordance with the embodiment of FIG. 1(a). However, other suitable configurations can be utilized. For example, FIG. 1(b) is another configuration to collect HE-HSI data from a transparent object like a histology slide. Light from the source 101 is directed to the bottom surface of the sample 102, passes through the sample 102, and is captured by the camera 103.

FIG. 1(c) shows the HE-HSI output from the camera of either FIG. 1(a) or FIG. 1(b). The 105 is a 4-dimensional data set that contains spectral information from both emission and excitation axes for all pixels within the observation field.

As shown in FIG. 1(c), the intensity values along λ1, λ2 axes (where λ1 is excitation wavelength and 22 is emission wavelength) stands for sample fluorescence. The values of diffuse reflectance are shown along the diagonal line. Since intensity of fluorescence is much less than intensity of reflected light, either camera must have a high dynamic range or there is a need for a device that can modulate the gain of the camera to adjust for the intensity of reflected light.

Each spectral plane corresponds to an image made from individual pixels each with xᵢ and yᵢ spatial coordinates. Each pixel contains information about two spectral axes or what is known as EEM (excitation-emission matrix). Together, two spatial and two spectral axes create a 4D HE-HSI dataset. The processing device can then determine, for example, a specific composition of sample material within each pixel.

One advantage of the HE-HSI system is that it acquires the full excitation-emission matrix (EEM) fluorescence data for each pixel in an image, rather than just a single excitation or emission wavelength as in traditional HSI. This provides much more spectral information to enable better identification and characterization of different tissue types, materials, etc.

In addition, the system 100 can collects the EEM data, in near real-time, by using fast tunable filters or tunable light sources and snapshot hyperspectral cameras. This enables practical use in surgical and other applications where quick acquisition is needed. The system 100 also acquires both fluorescence EEM data as well as reflectance HSI data for each pixel. The combination provides complementary information that improves detection over either modality alone.

The rich 4D dataset enables advanced computer analysis and machine learning to identify spectral signatures of interest. This avoids the need to pre-determine optimal excitation/emission wavelengths. The comprehensive spectral data can help overcome issues with inter-patient variability in tissue optical properties that can confound traditional HSI approaches. Once key spectral signatures are identified, the system can help design simpler, customized devices just using the specific wavelengths of interest for a given application.

FIG. 2(a) shows an example setup 200 optimized for fast acquisition. The optical path can have insertable components or switch between the two optical paths 1 and 2 shown. The reflectance spectra are acquired all at once using a snapshot camera 203 and a broadband source with pair of polarizers 205, 206 to remove specular reflectance. The fluorescence part of the spectra is then added by engaging a tunable light source and increasing the gain of the snapshot camera 203. Oversaturated images at wavelengths close to the excitation wavelengths are then replaced by the reflectance data to obtain the complete EEMs. In some cases, the reflectance data might not be necessary with EEM data containing only fluorescence spectra being highly informative.

As shown in FIG. 2, the imaging system 200 includes a lens system 207, tunable and/or broadband light sources 201a, 201b, an imaging device (e.g., snapshot hyperspectral camera) 203, and a polarizer pair formed by a first polarizer 205 and a second polarizer 206. The HE-HSI imaging system 200 is shown to determine properties or features of a sample 202, for example a specimen (e.g., tissue), material or object. The sample 202 can be a fixed sample or live sample, biological or non-biological, or other suitable samples.

An alternative to having an additional optical path 2, a filter 208 can be inserted after of light source 201a to illuminate the sample with different excitation wavelengths. In those embodiments, a broadband light source 201a transmits light to the tunable fast filter 208. The light source can range from a simple tungsten-halogen lamp to a super continuum white light laser. The light source can be optimized or chosen to operate in the ultraviolet (UV, 100-380 nm), visible (380-780 nm), short-wave infrared (SWIR, 780-3000 nm), and mid-/long-wave infrared (MWIR/LWIR, 3000-14000 nm).

The filter, here shown as a tunable bandpass filter 208, receives the light from the broadband light source 201a. The fast-bandpass filter 208 can be based on a rotatable multi-layered thin film optical filter, liquid crystal tunable bandpass filter, monochromator such as prism or diffraction gratings, or emerging technologies based on meta materials, epsilon-near-zero photonics, phase change materials, micro-electromechanical systems, photo-responsive hydrogels, etc. For currently available commercial fast tunable bandpass filters, switch time is about 5-50 msec or 0.5 ms/nm tuning speed. The filter 108 outputs a filtered light through optical path 1.

The light filter 208 is used to select specific wavelengths from broadband light. The broadband light contains a wide range of wavelengths across the electromagnetic spectrum. The light filter 108 allows only certain desired wavelengths to pass through while blocking other wavelengths. The system 200 can utilize any suitable system and method to filter light to selects wavelength(s), such as, e.g., computer tuning, mechanical tuning, and mechanically rotating LEDs. The transmission of the filter can be electronically controlled and rapidly tuned via computer to select whatever wavelengths are needed. This allows the selected wavelengths to be changed quickly and precisely through software. For mechanically rotatable filters, the filter can contain a set of distinct filter elements that each transmit different wavelengths. By mechanically rotating the filter, different filter elements can be moved into the light path to select different wavelengths.

For mechanically rotating LEDs, instead of a filter, an array of LEDs emitting at different wavelengths can be used. By mechanically rotating the LED array, LEDs emitting the desired wavelengths are rotated into the light path.

The specific wavelengths transmitted by the filter depend on the sample being analyzed and the application. For example, certain wavelengths may be useful for detecting a particular chemical in a sample. The filter 208 can be tuned or rotated to transmit only those useful wavelengths while blocking all others. This helps isolate the wavelengths of interest for specific application or target tissue.

For acquisition of reflected light, a pair of polarizers 205, 206, are inserted in the light path to avoid direct reflection artifacts that are otherwise commonly observed when the surface of a sample is not perfectly flat. Accordingly, the filtered light is received by the first polarizer (or input polarizer) 205. The first polarizer 205 outputs a polarized light 3. The first polarizer 205 operates as a filter that only allows light waves oscillating in one specific plane to transmit through while blocking light waves oscillating in other planes. The first polarizer 205 can be, for example, a material such as plastic or glass that has been treated with a special alignment process. This material alignment causes the electric field component of incoming light to orient in the alignment direction as it passes through. This produces an output beam that oscillates along a single axis, referred to as linearly polarized light. If linearly polarized light undergoes specular reflection the reflected light remains linearly polarized.

A properly oriented second polarizer (output polarizer) 206 can effectively filter specular reflections if placed at a proper angle to the axis of the polarizer 205. By putting two polarizers 205, 206 at correct angles to each other, the transmission of specular reflected light can be blocked by the second polarizer 206. In contrast, the second polarizer 206 does not efficiently filter out diffuse reflectance, since the latter has multiple randomly oriented polarization planes.

The lens system 207 is optionally provided. The lens system 207, can include, for example, an assembly of one or more optical components, such as optical fibers, photographic lens, endoscope, fundus camera, microscope objectives. One or more components of the lens system 207 receive the polarized light 3 from the polarizer 205 and direct it to the sample 202. The lens system 207 is configured based on target size, distance, and accessibility of the sample 202.

The light reflected from the surface of the sample 202 has two components, namely specular and diffuse reflectance. The specular component is effectively blocked by the properly oriented second polarizer 206. The diffuse reflectance component can pass thru the second polarizer 206 and travel toward the camera sensor 203.

The reflected light 4 is optionally received by one or more components of the lens system 104. As noted, the lens system 207 can have one or more optical components, and the reflected light can be directed by the same or different optical components as the polarized light 3. The one or more components of lens system 207 directs the reflected light 4 to the second polarizer 206.

To capture spectral planes at once the imaging device 203 can be a snapshot HSI camera. Traditional scanning hyperspectral cameras require multiple images of the same scene to be captured at different wavelengths sequentially. However, snapshot hyperspectral cameras capture all wavelengths simultaneously. This makes them much faster and more efficient for capturing hyperspectral data. The camera 203 can be based on a Fabry-Perrot interference filter array on top of a fast CMOS sensor, or any other technology used to split spectral bands into different spatial fields. The imaging device 203 can be any suitable imaging device, such as, for example, available commercial HSI cameras can collect 16 spectral bands hyper dataset for <3 msec. The imaging device 203 outputs an image array 209.

FIG. 2(a) shows the overall HE-HSI system in which reflectance and fluorescence data are acquired sequentially. FIG. 2(b) shows the first step of this process to collect reflectance data. FIG. 2(c) shows the second step of the process to collect fluorescence data. By combining these datasets, a complete EEM is created for each pixel of an image.

FIG. 2(a) is a general representation of the overall approach that depicts the key components; whereas FIGS. 2(b), 2(c) show how full spectrum HE-HSI is acquired using the image system 200 of FIG. 2(a). The first step is shown in FIG. 2(b), where the reflectance part of data of HE-HSI dataset is acquired yielding the diagonal line on the EEM image. Any suitable configuration can be utilized, such as a traditional HSI approach based on diffuse reflectance yielding 3D dataset with spectral coordinate λ1 (excitation) being equal to λ2 (emission). Broadband light 1 is transmitted from the light source 201a directly to the first polarizer 205, then the polarized light is transmitted to one or more components of the lens system 207. Thus, the first polarizer 205 is positioned between the light source 201a and one or more optical components of the lens system 207. The reflected light passes by or through one or more components of the lens system 207 to the second polarizer 206 to the camera 203. Thus, the one or more optical components of the lens system 207 is positioned between the second polarizer 206 and the sample, and the second polarizer is positioned between the camera 203 and the one or more components of the lens system 207.

The second step is shown in FIG. 2(c), where the rest of the 4-dimensional HE-HSI dataset is acquired to provide fluorescence data. Instead of a broadband light source 201a, a tunable light source 201b is now engaged in the light path. The tunable light source 201b can be replaced by the broadband light source 201a in combination with an insertable tunable filter 208. The tunable light source 201b directs excitation light through one or more lenses 207 toward the sample 202, while lens 207 directs light toward the camera 203. A polarizer pair is not necessary since there is no specular reflection. The camera 203 captures fluorescence spectra.

The reflectance spectra from Step 1 (FIG. 2(b)) can then be combined with the fluorescence spectra from Step 2 (FIG. 2(c)) to obtain a complete EEM for each pixel of an image.

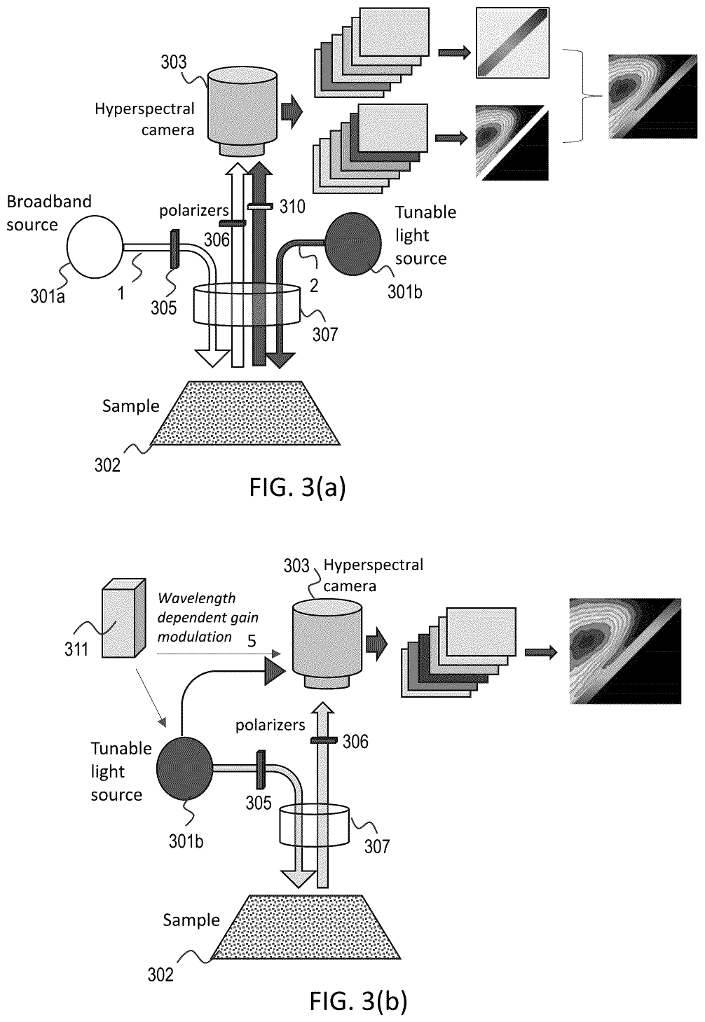

FIGS. 3(a), 3(b) are a HE-HSI image system 300 without the need for fast acquisition, therefore there is no need to use snapshot hyperspectral camera for the imaging device 303 (as in FIGS. 2(a)-(c), which use a snapshot hyperspectral camera 203) and the acquisition of the individual spectral planes occurs sequentially by a spectral hyperspectral camera for the imaging device 303. FIG. 3(a) shows HE-HSI setup when the reflectance and fluorescence data are acquired in two stages while FIG. 3(b) shows the setup that allows to acquire them in one passing.

The optical path in FIG. 3(a) can be designed to have insertable components or to switch between the two paths shown. The reflectance data can be acquired using a traditional spectral HSI camera or a regular black/white camera with a tunable filter in front of it. A broadband source 301a with a pair of polarizers 305, 306 can be used to remove specular reflectance. The fluorescence part of the spectra is then added by engaging tunable light source 301b and increasing the gain of the camera. Oversaturated images at wavelengths close to the excitation wavelengths are then can be replaced by the reflectance data to obtain the complete EEMs. Alternatively, a tunable band-pass, long-pass filter or dichroic mirror 310 can be used to let only the wavelengths above the excitation wavelength to pass toward camera 303.

FIG. 3(b) shows another embodiment that employs a single excitation tunable light source with wavelength-dependent gain modulation. This will enable to reduce camera gain when excitation wavelength is getting close to the emission wavelength. The gain signal 5 can, for example, be provided by the processing device 311 that is in communication with the imaging device 303, the filter 308, and/or light source 301a, whereby the processing device 311 controls both the imaging device 303 and the tunable source of excitation light. Such gain modulation will only be required for sequential acquisition of spectral planes (i.e., not snapshot cameras when all spectral planes are acquired at once). The gain modulation signal 5 adjusts for large differences in intensity of reflected versus fluorescent light, a wavelength-dependent sensor gain modulation can be implemented. The camera gain is automatically decreased, by the processing device, when light is collected at wavelengths in the vicinity of the wavelength of the illuminating light. Accordingly, the processing device 311 can adjust the sensitivity of the camera 303 based on the wavelength of the light coming from the source 301b. It is further noted that cameras with very large dynamic ranges can eliminate the need for gain modulation altogether.

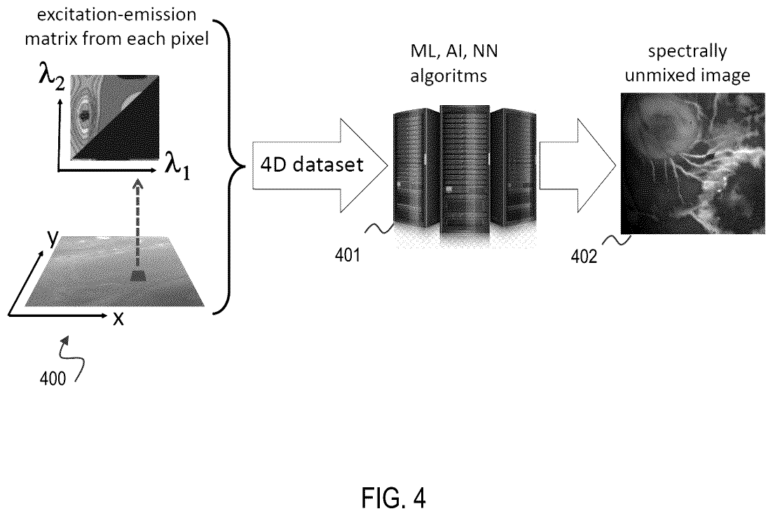

FIG. 4 shows a general concept of HE-HSI data processing that includes HE-HSI datasets 400, a processing unit 401, and the outcomes of spectral unmixing 402 that display different sample components using pseudocolors.

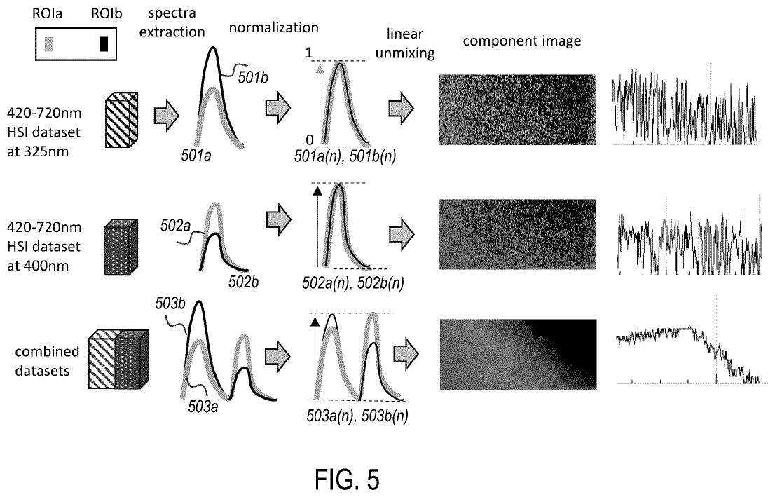

FIG. 5 shows, using an extremely simplified case of just two excitation wavelengths, an example of the HE-HSI principle. A small rectangular piece of a cellulose-based material was illuminated sequentially with two different wavelengths of UV light (325 nm and 400 nm in this case), at two different ROIs. At each illumination setting, a hyperspectral dataset was acquired from 420 nm to 720 nm in 10 nm spectral steps, which was then normalized and unmixed. Linear unmixing is a spectral unmixing technique which assumes that the spectral response of each pixel can be represented by a linear combination of the spectra of pure endmembers present in the scene. It then estimates both the endmembers and their fractional abundance maps, allowing for the identification and quantification of different materials in the image. Linear unmixing of either dataset alone, whether by automatic PCA or region-of-interest (ROI)-based unmixing, did not resolve the underlying spectral gradient. However, when the datasets are combined and spectra are extracted from the same ROIs, the gradient was clearly revealed. The reason for improved ability to recognize different materials is the larger amount of information contained within HE-HSI datasets.

FIG. 5 provides a graphical explanation of how the use of multiple excitation wavelengths can be more powerful compared to traditional HSI based on either single wavelength excitation or single wavelength emission. During typical HSI dataset processing steps, the raw spectra extracted from ROIs or individual pixels are normalized (i.e., stretched from 0 to 1). Therefore, differences in the amplitudes of individual spectral peaks can be lost. In the shown example, under 325 nm illumination, the spectral profiles from the two ROIs are very similar, but the amplitude is lower for the gray ROI. The situation reverses for 400 nm illumination. In both cases, normalization of an individual HSI dataset leads to nearly identical spectral profiles for the two ROIs. However, when the two HSI datasets are combined, the normalization step now stretches the spectra in a way that accentuates the differences between the ROIs. This yields superior unmixing results, as shown in the image and intensity profile on the right.

The simplified example shown here uses just two excitation wavelengths, but any suitable number of wavelengths can be provided such as for example thirty wavelengths, all of which can be combined. By acquiring spectral data from multiple combinations of wavelengths along excitation or emission axes enables to reveal additional details and/or minute differences in sample composition.

To create FIG. 5, a sample was first illuminated with 325 nm (the top row of FIG. 5) and a HSI dataset of 31 spectral bands (from 420-720 nm at 10 nm spectral step) was acquired. A spectral profile 501a was then extracted from the region of interest (ROIa, grey thick line), and another spectral profile 501b from the ROIb (black thin line). Next, the same sample was illuminated with the second excitation wavelength of 400 nm (the middle row of FIG. 5) and the second HSI dataset was acquired. The spectral profile 502a was then extracted from the ROIa (grey thick line), and another spectral profile 502b from the ROIb (black thin line).

The extracted spectral profiles 501a, 501b, 502a, 502b were then normalized for each ROI. The normalized spectral profiles from 325 nm HSI dataset for the ROIa and ROIb are labelled 501a(n) and 501b(n) respectively. The normalized spectral profiles from 400 nm HSI dataset for the ROIa and ROIb are labelled 502a(n) and 502b(n) respectively.

At the last row of FIG. 5, the same procedure is shown but for a new HSI dataset that was made by combining 325 nm HSI and 400 nm HSI datasets. The raw spectral profiles 503a and 503b are shown, followed by the normalized spectral profile 503a(n) and 503b(n).

The panel on the right shows the component images based on linear unmixing of all three HSI datasets shown on the left using the corresponding spectral profiles extracted from ROIa and ROIb. As illustrated, the linear unmixing of the last HSI dataset that was combined from two HSI datasets that used different excitation wavelengths clearly indicate a change in the sample property or characteristic that was not revealed by the linear unmixing of HSI datasets based on individual excitation wavelengths.

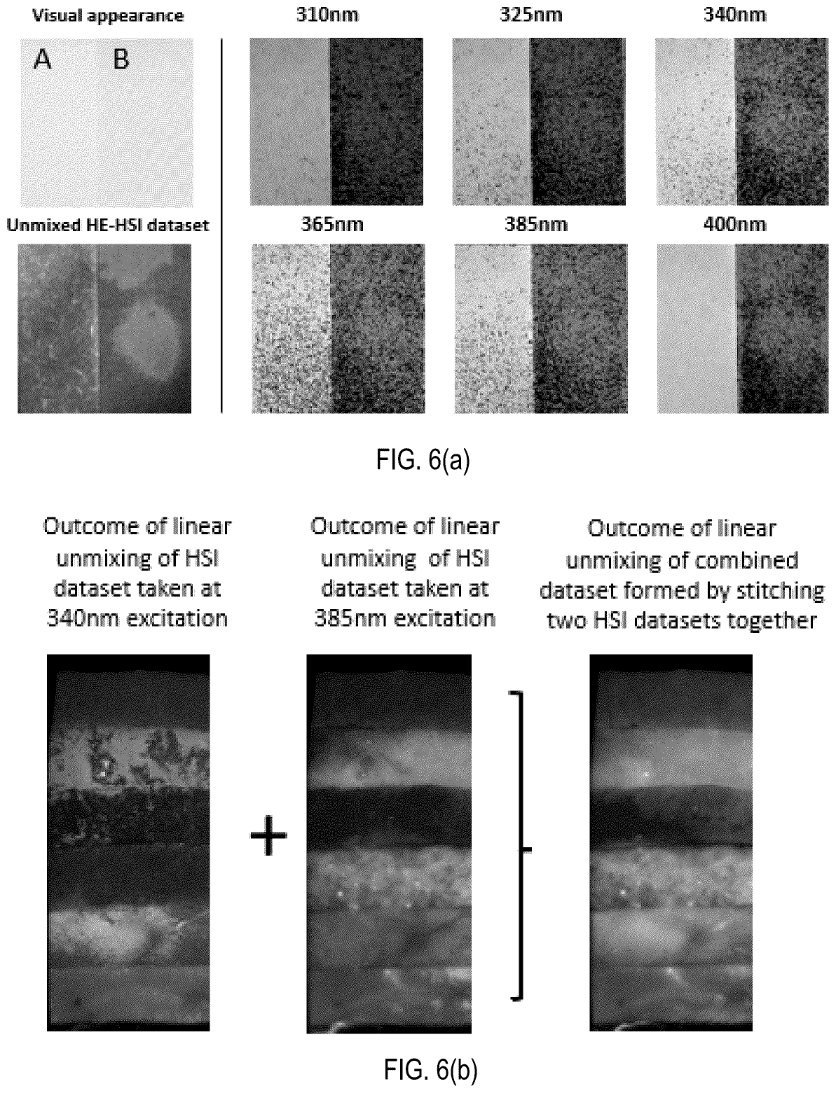

FIGS. 6(a) and 6(b) illustrate the increased sensitivity of the HE-HSI system versus a traditional, single wavelength excitation approach. The example shown in FIG. 6(a) is a sample made from strips of two different white paper materials. The strip B on the right has dried drops of diluted casein solution that are invisible to a human eye. Unmixing individual HSI datasets taken at six different excitation wavelengths do distinguish between the two types of the paper, yet it fails to reveal any additional information. In contrast, by combining six HSI datasets into a single 4D array with an additional axis for excitation wavelengths, reveals casein drop pattern on the paper strip B and additional inclusions within paper material A.

The example shown in FIG. 6(a) is a sample made from six strips of different deli meats. Similar to the examples shown in FIG. 5 and FIG. 6(a), the use of second excitation wavelength improves the outcomes of spectral unmixing.

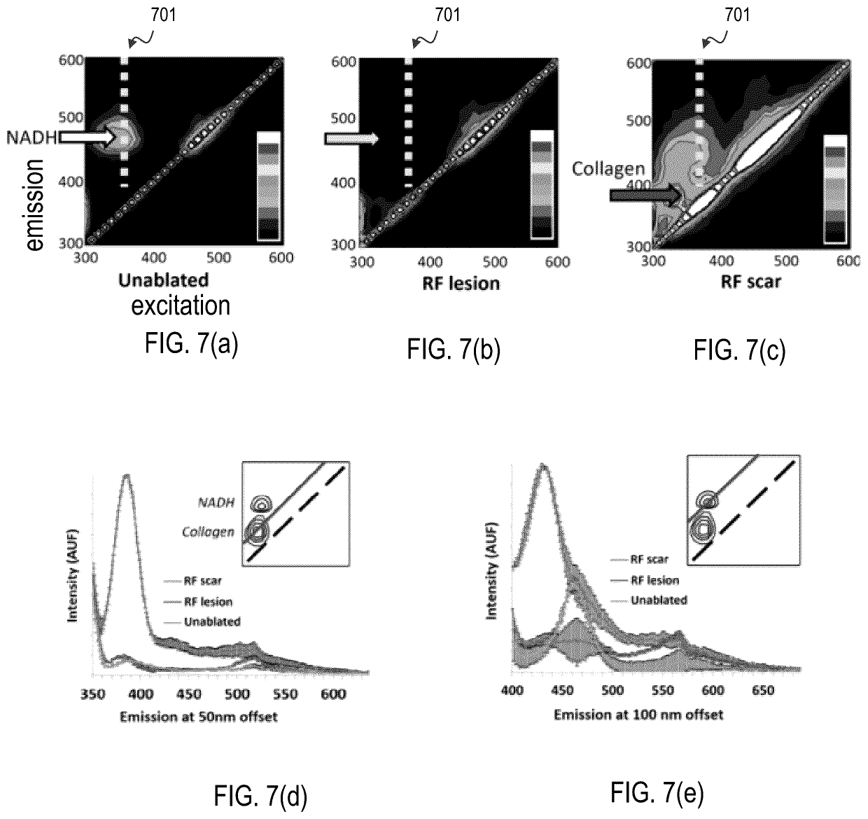

The EEMs shown in FIGS. 7(a)-7(c) are a further example of how information from HE-HSI datasets can be used. The dotted lines labelled 701 correspond to the area of the EEM that the traditional HSI cameras will collect from 420-720 nm emission range using 360 nm excitation settings. Since the HE-HSI system collects intensity information from the entire EEM area, it can aid designing protocols that use different offsets between the excitation and emission wavelengths. The examples shown in FIG. 7(d) and FIG. 7(e) illustrates that the three types of tissue (unablated, RF ablated and post-RF scarred myocardium) can be distinguished spectrally much better using 100 nm offset values compared to 50 nm offset values (solid red lines).

### Data Analysis

Referring to FIG. 4, the first part of the HE-HSI systems 100, 200 and 300 is the acquisition of 3D and 4D data (401). The second part of the HE-HSI system is data processing and display of final patterns (402). As shown, a data processing device 401 receives the data 400 from the camera, analyzes that data using Machine Learning (ML), Artificial Intelligence (AI), and neural network (NN) algorithms, and generates spectrally unmixed component images 402. The EEMs shown in FIG. 1(c) represents information from both spectral axes (21, 22 that stand for excitation and emission respectively) for each pixel leading to HE-HSI 4-dimensional data set with 4 axes (x, y, λ₁, λ₂).

One of the major challenges of traditional hyperspectral imaging is the need to identify specific excitation or emission wavelengths where spectral differences are consistent and/or pronounced. The hyperexcitation hyperspectral imaging (HE-HSI) systems 100-300 overcome these challenges by illuminating and acquiring the target surface with multiple wavelengths across the entire UV-VIS-IR range, while acquiring both fluorescence and reflectance profiles from each pixel of an image. Illumination with different wavelengths of light can be accomplished by using tunable lasers, switchable LEDs, or a combination of broadband light sources with filters. The latter can be a filter wheel, a prism, a diffraction grading, a liquid-based, a piezo-based or other types of tunable optical filters. UV-VIS-NIR covers the spectral range from around 100-1400 nm which includes the majority of optical imaging techniques. By acquiring hyperspectral data across this broad range, the HE-HSI system can capture a very comprehensive spectral profile for each pixel in an image frame. This enables sensitive and detailed analysis of the chemical composition and structure of samples. UV: 200-400, VIS: 400-700, IR: 700-1400 nm. Hyperspectral data can be collected with spectral steps between excitation and/or emission wavelengths of a range of sizes, for example sizes from 1 to 20 nm, preferably 5 nm or 10 nm.

The massive amount of collected data presents a rich source of spectral information that can be then analyzed by a variety of advanced image processing and machine learning algorithms, at the processing device 401 (FIG. 4).

Applications of the HE-HSI systems 100-300 includes multiple clinical targets including surgery of internal organs, dental applications, dermatological procedures, cancerous lesion identification, or any other type of medical procedure where one suspects changes in the spectral profile of the tissue. The HE-HSI systems can also be extended to non-clinical applications to reveal features otherwise invisible to the human eye, such as for example food processing, recycling or detection of art forgery. Similar to RGB images having more information than grayscale, the 3D HSI datasets are more informative than RGB, and the 4D HE-HSI data arrays are more informative than the 3D HSI dataset (FIG. 5-7). Therefore, when it comes to revealing the subtle spectral differences between targets of interest, one expects that the HE-HSI approach should have higher sensitivity and specificity when compared to greyscale, RGB, and traditional HSI.

For clinical applications where time is important, the HE-HSI system shown in FIG. 2 can provide real-time or near real-time imaging. From an acquisition viewpoint, the fast tunable excitation filter 208 can be any suitable filter, such as a filter that shifts to another center wavelength within 0.5-5 msec or faster. (See www.semrock.com/versachrome-edge-tunable-filters.aspx; www.thorlabs.com/newgrouppage9.cfm?objectgroup_id=3488; Wu J, Xie Z T, Sha Y, Fu H Y, Li Q. Epsilon-near-zero photonics: infinite potentials. Photonics Res 2021; 9:1616; Favreau P F, Rich T C, Prabhat P, Leavesley S J. Tunable thin-film optical filters for hyperspectral microscopy. Three-Dimensional and Multidimensional Microscopy: Image Acquisition and Processing XX. 2013. p85890R). In addition, snapshot hyperspectral cameras can be utilized that acquire multiband hyperspectral datasets on the order of a few msec. (See https://www.ximea.com; https://www.bayspec.com/spectroscopy/goldeneye-snapshot-hyperspectral-camera/). Therefore, combining, for example, Kurios® Liquid Crystal Tunable Bandpass Filters from Thorlabs with XMEA Hyperspectral Snapshot USB3 will yield 320 x-y spectral planes (20 excitation*16 emission wavelengths) in 60 msec. As used in this disclosure, the terms “real time,” “substantially real time,” and “near real-time” generally indicate that the processing and display of hyperspectral data is fast enough to facilitate visual perception of movement. For most clinical applications, a timeframe between 30 ms and 1000 ms can be considered “near real time”.

The second part of the HE-HSI systems includes data processing and display of final patterns (401, 402). Multiple real-time or near-real-time algorithms have been proposed for fast processing of HSI datasets making the HE-HSI approach suitable for many clinical applications, surgery being one example. (See Tournier J D, Smith R, Raffelt D, Tabbara R, Dhollander T, Pietsch M, Christiaens D, Jeurissen B, Yeh C H, Connelly A. MRtrix3: A fast, flexible and open software framework for medical image processing and visualisation. Neurolmage).

In addition, once full-range HE-HSI datasets are acquired and analyzed for a specific application, it allows for the identification of narrower spectral ranges along either of the two wavelength axes. Fewer number of wavelengths will then enable to speed up both acquisition and processing steps.

Furthermore, once the specific EEM profiles of interest are identified, the processing algorithms can sort pixels based on EEM sorting/matching to pre-established EEM libraries. The latter process is much faster than the de-novo spectral analysis of spectral profiles.

The HE-HSI system 100 overcome several shortcomings of traditional HSI. First, HSI can yield useful results only after someone spends significant time and effort in figuring spectral ranges that are unique to that specific pathology or condition. In other words, for example, to document melanoma progression, one needs to use light sources and HSI settings different from the protocols that aim to outline ablation lesions on the atrial surface. The use of HE-HSI systems 100-300 enables one to employ the same device for a variety of tissues and targets while letting subsequent processing algorithms detect spectral differences.

Secondly, data from biological tissues are notoriously noisy, therefore HSI settings might not work for samples from other body areas, animals, or individuals. Acquiring the entire EEM from each pixel will increase the richness of spectral datasets, helping to minimize the effect of this intrinsic variability of biological tissues. This is particularly important when it comes to the design of imaging devices to be used clinically (detailed next). When the number of photons returning to the sensor decreases, signal noise and spectra variability increase, lessening HSI's ability to reveal the desired targets.

Such a decrease in the number of returning photons inevitably occurs when one transitions from on-the-bench studies to producing an actual clinical HSI device. This is because most clinical targets must be observed percutaneously or endoscopically, so the camera sensor must be coupled to a fiberoptic assembly within the body of the catheter or an endoscope. This greatly decreases the returning photon count compared to on-the-bench studies (the latter is routinely done by outfitting HSI cameras with high numerical aperture objectives). In addition, in clinical settings, the data needs to be acquired in seconds rather than minutes, again dramatically decreasing the amount of light going back to the sensor (See Armstrong K, Larson C, Asfour H, Ransbury T, Sarvazyan N. A Percutaneous Catheter for In Vivo Hyperspectral Imaging of Cardiac Tissue: Challenges, Solutions and Future Directions. Cardiovasc Eng Technol 2020; 11:560-575).

By expanding HSI to HE-HSI systems 100-300, these major shortcomings for the use of HSI percutaneously or endoscopically can be ameliorated. Additional spectral information might compensate for a poor signal-to-noise ratio seen observed for low light samples or conditions.

Thirdly, once full-range HSI datasets are acquired and analyzed for a specific application, the spectral ranges can be narrowed along either of the two wavelength axes. Using fewer wavelengths enables to speed up both the acquisition and processing steps.

Fourthly, once the specific EEM profiles of interest are identified, the processing algorithms can sort pixels based on EEM matching to pre-established EEM libraries. The latter process is much faster than the de-novo spectral analysis of spectral profiles. For most clinical targets, however, it might be not critical to get HE-HSI results at a video rate, immediate examples being dermatology or dentistry. Even during surgery of internal organs, it should not be a problem to hold the HE-HSI device over the surface of interest for several seconds to get a clear delineation of the tumor area, ablated tissue, or to obtain better vessel outlines.

The HE-HSI systems can be utilized to develop application-specific spectral devices. The HE-HSI systems can pinpoint specific spectral regions/settings effective for a particular clinical application. In this case, the initial, high spectral resolution HE-HSI datasets can be acquired without any time constraints. Let's consider a hypothetical example related to dentistry, specifically to documenting changes in the shade of gums affected by lichen planus. The initial, spectrally detailed HE-HSI datasets can take several minutes to acquire (i.e., the research stage of the study). Subsequent data analysis can then pinpoint a few spectral ‘spots’ where spectral changes are the most pronounced. This means that instead of, for example, 100×100≈10,000 spectral planes to acquire and process, one will need only 3. Based on this information, a much simpler, faster, and cheaper device can be developed for this particular clinical target.

Additional lines of evidence to support the clinical potential of HE-HSI approach come from studies that compared the use of excitation-based HSI to emission-based HSI. Each of these two sub-modalities presented certain advantages, therefore by combining them into HE-HSI approach these advantages will be amplified (See Favreau P F, Hernandez C, Heaster T, Alvarez D F, Rich T C, Prabhat P, Leavesley S J. Excitation-scanning hyperspectral imaging microscope. J Biomed Opt 2014; 19:46010; Leavesley S J, Walters M, Lopez C, Baker T, Favreau P F, Rich T C, Rider P F, Boudreaux C W. Hyperspectral imaging fluorescence excitation scanning for colon cancer detection. J Biomed Opt 2016; 21:104003; Zuluaga A F, Utzinger U, Durkin A, Fuchs H, Gillenwater A, Jacob R, Kemp B, Fan J, Richards-Kortum R. Fluorescence excitation emission matrices of human tissue: a system for in vivo measurement and method of data analysis. Appl Spectrosc 1999; 53:302-311).

Furthermore, studies show that by combining information from fluorescence and reflectance HSI data, additional information is to be gained (Chang S K, Mirabal Y N, Atkinson E N, Cox D, Malpica A, Follen M, Richards-Kortum R. Combined reflectance and fluorescence spectroscopy for in vivo detection of cervical pre-cancer. J Biomed Opt 2005; 10:24031; Noh H K, Peng Y, Lu R. Integration of hyperspectral reflectance and fluorescence imaging for assessing apple maturity. Trans ASABE 2007; 50:963-971; Asfour H, Aljishi M, Chahbazian T, Swift L M, Muselimyan N, Gil D, Sarvazyan N A. Comparison between Autofluorescence and Reflectance-Based Hyperspectral Imaging for Visualization of Atrial Ablation Lesions. Biophys J 2016; 110: 493a-494a). The HE-HSI relies on a combined analysis of diffuse reflectance and fluorescence spectra which ensures that the potential benefits of using either modality will not be omitted.

Data from biological tissues are notoriously noisy, and the signals are variable. Acquiring the entire EEMs from each pixel and accounting for inter-pixel correlation increases the richness of spectral datasets and helps to minimize the effect of intrinsic variability of biological samples.

To better illustrate the richness of prospective HE-HSI datasets we are showing three EEMs acquired using single-point spectrofluorimetry from different areas of treated and untreated rat epicardial surface (FIGS. 7(a)-7(c)). The dotted line 701 corresponds to the area of the EEM that the HSI cameras collects at 360 nm excitation settings. One can clearly see that the autofluorescence information contained within the entire EEM is much richer than the data contained along the yellow dotted line (i.e., emission profile at 360 nm excitation).

Moreover, in addition to fluorescence data, EEM datasets also contain diffuse reflectance profiles. Specifically, the diagonal line on EEM corresponds to λ_{excitation}=λₑₘᵢₛₛᵢₒₙ, or the degree by which light of specific wavelength is being reflected by the sample surface (due to two-order difference in the intensity of returning light, to display EEM fluorescence and reflectance data one must use different LUT scales). FIGS. 7(d)-7(e) show the use of EEM datasets to create simpler, application-specific HE-HSI protocols or devices. Specifically, it shows spectral profiles derived from the EEMs above using different offsets between the excitation and emission wavelengths. The three types of tissue can be distinguished much better using 100 nm rather than 50 nm offset values (solid red lines).

FIGS. 7(a)-7(e) show that spectral information acquired using the HE-HSI system can be used to provide application-focused, faster and/or simpler imaging protocols. Three EEMs were acquired from three sites on a heart surface: a healthy tissue (FIG. 7(a)), an acute radiofrequency (RF) lesion (FIG. 7(b)) and a scar (FIG. 7(c)) formed at the site of healed RF lesion. By using different offsets between excitation and emission wavelengths the three conditions can be separated with great accuracy. In contrast, the traditional HSI will limit data acquisition to a single line across the EEM space (see dotted line).

### Applications

There exists a wide range of options for implementing HE-HSI systems shown in FIGS. 1-3. The key components include a tunable light source or a combination of a light source and a tunable filter. This enables the selection of specific illumination wavelength bands. Another key components are HSI cameras, which can capture multiple spectral planes in a simultaneous or sequential manner.

There are then a variety of methods for combining illumination and imaging optics to enable the system to image the target media and acquire both fluorescence and reflectance without operator intervention. Total HE-HSI capture time will depend on illumination source tuning speed and camera sensor integration time, which will depend on target irradiance.

The near real time HE-HSI system would provide sufficient illumination power at multiple wavelengths in order to minimize integration time. Fast tunable light sources will be able to minimize the time required to change illumination wavelength. Fast tunable filters such as liquid crystal and acousto-optical filters, can both tune in milliseconds, though the latter provides substantially higher optical efficiency.

Angle-dependent dichroic filters are available but would need to be stacked to provide a tunable bandpass solution. The light source can be multiple individual narrow-band sources, such as collimated LEDs, or a broadband source, such as a superbright white LED, xenon arc lamp, or supercontinuum laser, with a tunable filter. The most compact and cost-effective solution for illumination at multiple wavelengths is the use of stacked single-band LED light sources. In this case, the tuning speed would be limited by the slew rate of the LED current source, which would provide tuning times in the millisecond range.

The proliferation of drone-based surveillance and surveying has led to the increased availability of compact HSI cameras. Snapshot HSI cameras utilizing Fabry-Perrot interference filter arrays in combination with high-speed CMOS sensors are currently best suited for implementation of the HE-HSI approach. They provide numerous spectral bands across the UV-VIS-NIR range and capture times that depend on the total number of bands, with cameras providing rates as high as 6 ms per dataset.

Depending on the camera and light source optical interfaces, and the accessibility of the target media, light could be delivered and retrieved via imaging bundles, a custom endoscope or laparoscope, or a combination of photographic lenses. The acquisition of reflectance HSI data could be accomplished using the full bandwidth of the light source in combination with the addition of a polarizer pair to eliminate reflection artifacts from an irregular target surface. The collection of a complete set of EEM data, including both reflectance and fluorescence, by a sequential illumination with different wavelength requires either a high dynamic range from the camera sensor or modulation of gain at the excitation wavelength during capture. Alternately, reflectance data set could be collected at globally reduced integration time using the same wavelength of illumination, and the data subsequently scaled and combined.

In some embodiments, the HE-HSI spectral imaging systems 100-300, including the light source and camera, can be made portable for dermatological applications. The HE-HSI system can also be configured for non-invasive imaging of spectrally complex surfaces of human organs. The HE-HSI spectral imaging system can also include a HE-HSI-based endoscopic and percutaneous imaging device configured as surgical tools.

It is further noted that the processing device 401 can be implemented by a computer or computing device having a processor or controller to perform various functions and operations in accordance with the disclosure. The processing device can be, for instance, a personal computer (PC), server or mainframe computer, or be based on cloud computing. The processing device may also be provided with one or more of a wide variety of components or subsystems including, for example, a co-processor, register, data processing devices and subsystems, wired or wireless communication links, input devices, monitors, memory or storage devices such as a database. All or parts of the system and processes can be stored on or read from computer-readable media. The system can include computer-readable medium, such as a hard disk, having stored thereon machine executable instructions for performing the processes described. All or parts of the system, processes, and/or data utilized in the disclosure can be stored on or read from the storage device(s). The storage device(s) can have stored thereon machine executable instructions for performing the processes of the disclosure. The processing device can execute software that can be stored on the storage device.

The system and method of the disclosure can also be implemented by or on a non-transitory computer readable medium, such as any tangible medium that can store, encode or carry non-transitory instructions for execution by the computer and cause the computer to perform any one or more of the operations of the disclosure described herein, or that is capable of storing, encoding, or carrying data structures utilized by or associated with instructions.

The processing device can also be connected to or in communication with the Internet, such as by a wireless card. The processing device can interact with a website to execute the operation of the disclosure, such as to present output, reports and other information to a user via a user display, solicit user feedback via a user input device, and/or receive input from a user via the user input device. For instance, the processing device can be part of a mobile smart phone running an application (such as a browser or customized application) that is executed by the processing device and communicates with the user and/or third parties via the Internet via a wired or wireless communication path.

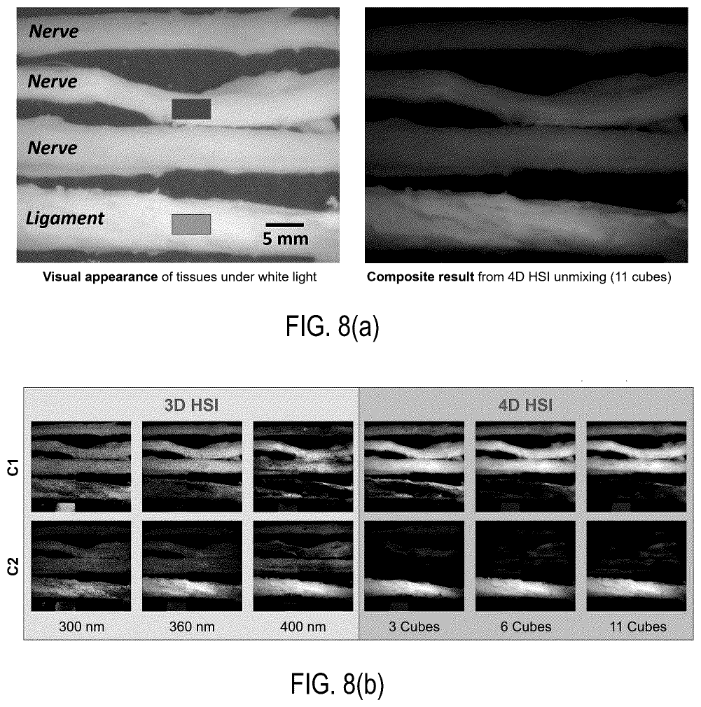

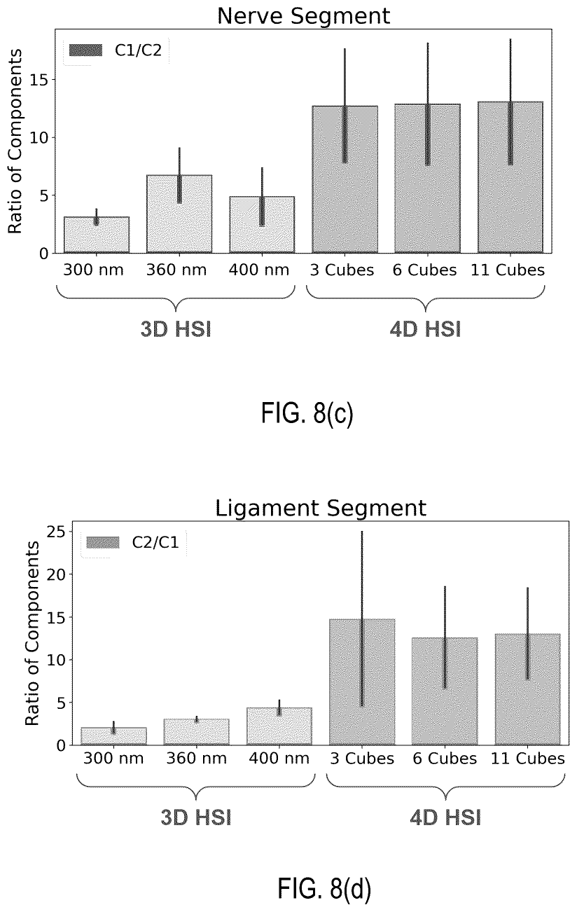

### Example Embodiment (FIG. 8)

FIG. 8 presents an example of applying HE-HSI approach vs. a traditional HSI. It serves as an illustration for visualizing clinically relevant targets in accordance with non-limiting embodiments of the disclosure. Details can be found in this recently presented work: N Chilingaryan, F Villarruel, T Soghomonyan, V Yeghiazaryan, N Sarvazyan. 4D hyperspectral imaging for intraoperative tissue classification. Proceedings of 2025 SPIE Medical Imaging, paper #13410-35, the entire contents of which are hereby incorporated by reference. Here we systematically compared autofluorescence-based HE-HSI to a traditional HSI approach for its ability to distinguish between nerve and ligament bundle which can help to avoid inadvertent nerve damage during surgery, the latter being result from inter-patient anatomical variability and visual similarity of nerve fibers to nearby structures. Dissected bovine tissues were used as examples of targets to be distinguished.

FIG. 8 shows the unmixing outcomes of HSI approach that uses only one spectral dimension (ie, 3D HSI with the other two axes being spatial coordinates x and y) versus HE-HSI approach that uses two spectral dimensions in addition to two spatial ones (therefore HE-HSI can be also called 4D HSI). 3D datacubes were captured within the 420-720 nm interval, under illumination wavelengths ranging from 300 to 400 nm. By combining multiple 3D HSI cubes, the 4D HSI sets were then derived.

In FIG. 8(a), the image on the left shows a visual appearance of tissues (three nerves, from top to bottom: lateral plantar nerve, medial plantar nerve, peroneal nerve) and the suspensory ligament of the fetlock. The rectangles are the respective ROIs based on which component 1 (C1) and component 2 (C2) spectra were unmixed. The pseudocolor image to the right is a composite image based on unmixing of a 4D HSI dataset composed of 11 individual 3D HSI cubes.

FIG. 8(b) shows grayscale component images where the relative intensity/quantity of individual component spectra per pixel that the unmixing algorithms have been able to separate the total signal into. FIG. 8(c) show mean intensities of the components within corresponding ratios between them. For all tested HE-HSI configurations, 4D HSI datasets overperformed the traditional 3D HSI-even as few as three 3D cubes were sequentially combined into one 4D dataset, FIG. 8(d).

More advanced image analysis techniques, such as convolutional neural networks (CNNs) can utilize data rich 4D HSI sets further to distinguish surgical targets (N Matosyan, M. N Chilingaryan, N Sarvazyan and V Yeghiazaryan. Combining 4D Hyperspectral Imaging with CNN for Nerve and Ligament Differentiation, 2025 IEEE 22nd International Symposium on Biomedical Imaging (ISBI); N Matosyan, N Chilingaryan, N Sarvazyan, V Yeghiazaryan. Spectral pixels as images: CNN-based pixel classification of 4D hyperspectral data for nerve and ligament differentiation. Proceedings of 2025 SPIE Medical Imaging, paper #13406-82), the contents of which are hereby incorporated by reference.

HE-HSI applications include a variety of clinical targets such as intraoperative monitoring of the surgical field of view during surgical treatment of internal organs, identification of ischemic areas, dental applications, detection of cancerous lesions, and other medical procedures where changes in the spectral properties of tissues are anticipated. Data presented in FIG. 8 confirm that the HE-HSI modality can reveal subtle differences between targets with significantly higher sensitivity than traditional excitation-only or emission-only HSI approaches.

The description and drawings of the present disclosure provided should be considered as illustrative only of the principles of the disclosure. The disclosure may be configured in a variety of ways and is not intended to be limited by the preferred embodiment. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

1. A hyperexcitation hyperspectral imaging system for analyzing a sample, comprising:
a light source sequentially illuminating the sample by different wavelengths of lights; and
an imaging device receiving light from the sample and simultaneously capturing multiple spectral planes at the different wavelengths of light.

2. A hyperexcitation hyperspectral imaging system for analyzing a sample, comprising:
a light source sequentially illuminating the sample by different wavelengths of lights; and
an imaging device receiving light from the sample and sequentially capturing multiple spectral planes at the different wavelengths of light.

3. The imaging system of claim 1, further comprising:
a first optical component configured to direct light from the light source to the sample; and
a second optical component configured to receive light from the sample and direct the light to said imaging device.

4. The imaging system of claim 3, wherein the first optical component is the same as the second optical component.

5. The imaging system of claim 1, wherein the first optical component is different than the second optical component.

6. The imaging system of claim 1, wherein said imaging device comprises a snapshot hyperspectral camera and acquires multiple spectral planes simultaneously from the light directed by said second optical component.

7. The imaging system of claim 2, wherein said imaging device comprises a hyperspectral camera capable of acquiring multiple spectral planes sequentially.

8. The imaging system of claim 1, wherein the light includes ultraviolet, visible and infrared light.

9. The imaging system of claim 1, wherein the four-dimensional data array created by said imaging device includes excitation and emission data for each pixel.

10. The imaging system of claim 1, further comprising a processing device configured to receive the data array from said imaging device and to provide spectrally unmixed image output.

11. The reflectance part of the imaging system of claim 1, further comprising:
a first polarizer configured to receive light from said light source and pass light oscillating in a specific desired plane to the sample; and
a second polarizer configured to receive light from the sample, block light oscillating in planes other than a specific desired plane, and pass light oscillating at the specific desired plane to said imaging device.

12. A hyper-excitation hyperspectral imaging (HE-HSI) spectral imaging system for use with a sample, the system comprising:
a tunable filter or tunable light source providing multiple illumination wavelength of light across the entire UV-VIS-IR range to a sample;
a hyperspectral camera receiving light from the sample and capturing images at multiple wavelengths of light; and
a processing device configured to analyze the reflectance, excitation and emission fluorescence spectra from each pixel of the image.

13. The HE-HSI spectral imaging system of claim 12, further comprising:
a broadband light source providing light to the sample;
an imaging device capturing light from the sample in response to the broadband light to provide reflectance spectra;
said processing device combining the reflectance spectra and the fluorescence spectra.

14. The HE-HSI spectral imaging system of claim 12, said processing device having an advanced data processing algorithm to identify targets previously indistinguishable by traditional hyperspectral imaging alone and reduce the amount of effort needed to find the optimal illumination and acquisition wavelengths.

15. The HE-HSI spectral imaging system of claim 12, wherein said HE-HSI system is portable for dermatological applications.

16. The HE-HSI spectral imaging system of claim 12, wherein said HE-HSI system is configured for non-invasive imaging of spectrally complex surfaces of human organs.

17. The HE-HSI spectral imaging system of claim 12, further comprising a HE-HSI-based endoscopic and percutaneous imaging device configured as surgical tools.
